(19) United States
(12) Patent Application Publication
(10) Pub. No.: US 2026/0325241 A1
(43) Pub. Date: Oct. 22, 2026

(54) MULTI-ARM SURGICAL ROBOTIC SYSTEM FOR TELE-SURGERY

(71) Applicant: SSI IP HOLDINGS INC., Ft. Lauderdale, FL (US)

(72) Inventors: Sudhir Prem Srivastava, Haryana (IN); Vishwajyoti Pascual Srivastava, Haryana (IN); Rama Krishna Reddy Dyava, Haryana (IN); Suraj Dwivedi, Haryana (IN); Shubhankar Sanjiv Kulkarni, Haryana (IN); Nitin Hemdan, Haryana (IN); Anson Paul, Haryana (IN); Munish Batra; Anil Kumar Patidar, Haryana (IN); S Naveen Ajay Kumar, Haryana (IN); Anuj Saini, Haryana (IN)

(21) Appl. No.: 19/108,058

(22) PCT Filed: Dec. 24, 2024
(86) PCT No.: PCT/IN2024/052438

§ 371 Date: Feb. 28, 2025

(30) Foreign Application Priority Data
Jan. 18, 2024 (IN) 202411003576

(51) Int. Cl. A61B 34/35; A61B 34/00; A61B 34/30; A61B 90/00

(52) U.S. Cl. CPC A61B 34/35; A61B 34/25; A61B 90/361; A61B 2034/258; A61B 2034/301; A61B 2034/306

(57) ABSTRACT

The invention relates to a multi-arm surgical robotic system for tele-surgery comprising a local system, a remote system, a private network, and a public network. The local system comprises of a plurality of robotic arms connected to an endoscopic camera, and the remaining robotic arms are each connected to one of a surgical robotic instruments, a local surgeon console coupled to a master controller to provide a control input to the plurality of robotic arms, and a conferencing system. The remote system comprises of a remote surgeon console coupled to a master controller and a conferencing system, which is operably connected to the conferencing system via the public network. The communication between the local surgeon console and the remote surgeon console via the private network, enables the remote surgeon to control and monitor the robotic arms, allowing the remote surgeon to perform a robotic surgical procedure remotely. In case of failure of the private network, control can be shifted from the remote surgeon console to the local surgeon console, allowing control of the robotic surgical procedure.

### CROSS REFERENCE TO RELATED APPLICATIONS

This application is a U.S. National Stage under 35 U.S.C. § 371 of International Application No. PCT/IN2024/052438 filed on Dec. 27, 2024, which claims priority to Indian Patent Application No. 202411003576 filed on Jan. 18, 2024, and entitled “A MULTI-ARM SURGICAL ROBOTIC SYSTEM FOR TELE-SURGERY”, which are incorporated herein by reference in its entirety.

It is intended that the referenced application may be applicable to the concepts and embodiments disclosed herein, even if such concepts and embodiments are disclosed in the referenced applications with different limitations and configurations and described using different examples and terminology.

### TECHNICAL FIELD

The present disclosure generally relates to a multi-arm robotic surgical system for minimally invasive surgery, and more particularly, the disclosure relates to a system and method for performing tele-surgery in a multi-arm robotic surgical system.

### BACKGROUND

This section is intended to introduce the reader to various aspects of art that may be related to various aspects of the present disclosure, which are described below. This disclosure is believed to be helpful in providing the reader with background information to facilitate a better understanding of the various aspects of the present disclosure. Accordingly, it should be understood that these statements are to be read in this light, and not just as an admissions of prior art.

Robotic assisted surgical systems have been adopted worldwide to gradually replace conventional surgical procedures such as open surgery and laparoscopic surgical procedures. The robotic assisted surgery offers various benefits to a patient during surgery and during post-surgery recovery time. The robotic assisted surgery equally offers numerous benefits to a surgeon in terms of enhancing the surgeon's ability to precisely perform surgery, less fatigue and a magnified clear three-dimensional (3D) vision of a surgical site. Further, in a robotic assisted surgery, the surgeon typically operates with a hand controller/master controller/surgeon input device/joystick at a surgeon console system to seamlessly capture and transfer complex actions performed by him/her giving the perception that he/she himself/herself is directly articulating a surgical tool/surgical instrument to perform the surgery.

The robotically assisted surgical systems may comprise of multiple modular robotic arms aiding in conducting robotic assisted surgeries. The surgeon controls the robotic arms, and the instruments mounted on it by using the surgeon console. The surgeon console comprises of visualization system to allow the surgeon to perform the surgery. Further, the hand controllers/the master controllers/the surgeon input devices are integrated with the surgeon console which the surgeon maneuvers to perform the surgery. The biggest challenge is the requirement for the presence of an expert surgeon in the operating room, which may not be possible due to the surgeon's prior commitments or geographical distance. Tele-surgery can solve this problem by allowing the surgeon operating on the surgeon console system to be located at a distance from a surgical site or within an operating room where the patient is being operated on.

Performing tele-surgery creates new challenges. The main challenge is the unavailability of a multi-arm robotic surgical system for performing tele-surgery. Further, another challenge is while performing a tele-surgery the surgeon commands from a remote location should be sent in a secured way to the robotic surgical system without interruption. Moreover, the existing systems do not allow the local surgeon to take over and the surgery needs to be converted to open in case of failure of transmission network.

Yet another challenge is that the information about the surgical instruments being utilized may not be known to the remote surgeon. Further, the 2D scans of the patient may not be available to the remote surgeon, who may like to check 2D scans of the patient before performing the surgery.

Also, sometimes, an inexperienced local surgeon may want to get guidance or proctoring from an expert remote surgeon. Further, the existing systems do not allow both the local and remote surgeon to control the robotic arms together.

In light of the aforementioned challenges there is a need for providing a multi-arm robotic surgical system which will solve the above-mentioned problems related to tele-surgeries.

### SUMMARY OF THE DISCLOSURE

Some or all of the above-mentioned problems related to performing tele-surgery in a multi-arm robotic surgical system are proposed to be addressed by certain embodiments of the present disclosure.

According to an aspect of the invention, there is disclosed a multi-arm surgical robotic system for tele-surgery, comprising a local system, a remote system, a private network, and a public network characterized in that: the local system comprises: a plurality of robotic arms arranged along an operating table, wherein a robotic arm out of the plurality of robotic arms is connected to an endoscopic camera, and the remaining robotic arms are each connected to one of a surgical robotic instruments; a local surgeon console comprising a left-hand controller, a right-hand controller, foot pedals, a two-dimensional (2D) touch screen monitor, a three-dimensional (3D) HD monitor, and a head tracking camera to track a movement of the head of a local surgeon wearing a pair of trackable glasses, each coupled to a master controller, the master controller provides a control input received from the local surgeon to the plurality of robotic arms; and a conferencing system, the remote system comprises: a remote surgeon console comprising a left-hand controller, a right-hand controller, foot pedals, a two-dimensional (2D) touch screen monitor, a three-dimensional (3D) HD monitor, and a head tracking camera to track a movement of the head of a remote surgeon wearing a pair of trackable glasses, each coupled to a master controller, wherein the master controller is operably coupled to the master controller of the local surgeon console via the private network; and a conferencing system operably connected to the conferencing system via the public network; the private network is configured to: transmit the control input provided by the remote surgeon to the master controller of the local surgeon console, and transmit a real-time, encrypted 3D video stream of the surgical site from the local surgeon console to the three-dimensional (3D) HD monitor of the remote surgeon console; retrieve DICOM images related to a patient from a hospital server connected to the private network and provide them to the master controller for displaying on the 2D display of the remote surgeon console and the master controller for displaying on the 2D display of the local surgeon console; and transmit the information related to the surgical instruments and the status of each robotic arms to the master controller to be displayed on the three-dimensional (3D) HD monitor, the public network is configured to facilitate a two-way transmission of real-time audio and video data between the conferencing system of the remote surgeon console and the conferencing system of the local surgeon console; wherein the communication between the local surgeon console and the remote surgeon console enables the remote surgeon to control and monitor the robotic arms, allowing the remote surgeon to perform a robotic surgical procedure remotely, wherein the control from the remote surgeon console can be shifted to the local surgeon console, allowing the local surgeon to assume control of the robotic surgical procedure in case of failure of the private network.

According to an embodiment of the invention, in the multi-arm surgical robotic system having a minimum five robotic arms, a partial control from the remote surgeon console can be shifted to the local surgeon console, enabling the remote surgeon to operate any three arms out of the five or more robotic arms and the remaining arms can be operated by the local surgeon acting as an assistant surgeon.

According to another embodiment of the invention, the left-hand controller, the right-hand controller, and the foot pedals are configured to capture the control input from the local surgeon.

According to yet another embodiment of the invention, the two-dimensional (2D) touch screen monitor coupled to the master controller is configured to serve as a graphical user interface to capture the control input from the local surgeon.

According to yet another embodiment of the invention, the real-time 3D video stream of the surgical site is displayed on the three-dimensional (3D) HD monitor of the local surgeon console.

According to yet another embodiment of the invention, the left-hand controller, the right-hand controller, and the foot pedals are configured to receive the control input from the remote surgeon.

According to yet another embodiment of the invention, the two-dimensional (2D) touch screen monitor coupled to the master controller is configured to be used as a graphical user interface to capture the control input from the remote surgeon.

According to yet another embodiment of the invention, each conferencing system comprises of a processor, a camera, a 2D monitor, a speaker, and a microphone.

According to yet another embodiment of the invention, the real-time two-way video data transmitted between the conferencing system of the remote surgeon console and the conferencing system of the local surgeon console can be displayed on separate 2D monitors at the remote location and the local operating room.

According to yet another aspect of the invention, a method of tele-surgery using a multi-arm surgical robotic system comprising a local system, a remote system, a private network, and a public network, the method comprising: establishing, using the private network, a communication link between the local system and the remote system, wherein the local system includes a plurality of robotic arms and a local surgeon console, and the remote system includes a remote surgeon console; transmitting, using the private network, a control input from a remote surgeon to the local system, wherein the control input controls the movement and operation of the plurality of robotic arms; providing, using the private network, a real-time encrypted 3D video of the surgical site from the local system to the remote system for visualization on a 3D HD monitor of the remote surgeon console; enabling, using a conferencing system of the local surgeon console and a conferencing system of the remote surgeon console via a public network, the real-time two-way audio and video communication between the local surgeon console and the remote surgeon console to facilitate remote collaboration during the surgical procedure; transmitting, using the private network, DICOM images of a patient from a hospital server for display on a 2D touch screen monitor of the remote surgeon console and on the 2D touch screen monitor of the local surgeon console; displaying, using the three-dimensional (3D) HD monitor of the local surgeon console and the three-dimensional (3D) HD monitor of the remote surgeon console, the information related to the status of the robotic arms and surgical robotic instruments; allowing, using the control input from the master controller, the control of the operation of the robotic arms from the remote surgeon console, wherein the remote surgeon is located distantly from the patient; shifting, using the remote surgeon console, the control from the remote surgeon console to the local surgeon console, thereby enabling, the local surgeon to assume direct control over the control of the operation of the robotic arms during the surgical procedure in case of failure of the private network; performing, using the robotic arms based on the control inputs from the remote surgeon, the surgical procedure on the patient, thereby enabling remote surgery.

According to still another embodiment of the invention, in the multi-arm surgical robotic system having a minimum five robotic arms, a partial control from the remote surgeon console can be shifted to the local surgeon console, enabling the remote surgeon to operate any three arms out of the plurality of robotic arms and the remaining arms can be operated by the local surgeon acting as an assistant surgeon.

Other embodiments, systems, methods, apparatus aspects, and features of the invention will become apparent to those skilled in the art from the following detailed description, the accompanying drawings, and the appended claims.

### BRIEF DESCRIPTION OF THE DRAWINGS

The summary above, as well as the following detailed description of the disclosure, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to the scale. Wherever possible, like elements have been indicated by identical numbers.

Embodiments of the present disclosure will now be described, by way of example only, with reference to the following diagrams wherein:

FIG. 1 illustrates an example implementation of multi-arm surgical robotic system for tele-surgery using a remote surgeon console in accordance with an embodiment of the disclosure;

FIG. 2 illustrates an example implementation of a multi-arm teleoperated robotic surgical system kept at the location of surgery in accordance with an embodiment of the disclosure;

FIG. 3 illustrates an implementation of a local surgeon console connected to a remote surgeon console via a private network in accordance with an embodiment of the disclosure;

FIG. 4 illustrates a flow of communication of control input from the remote surgeon console (140) to the local surgeon console (118) located in the local operating room in accordance with an embodiment of the disclosure;

FIG. 5(a) illustrates a block diagram indicating a communication of a control input from a remote surgeon at a remote location to local operating room in accordance with an embodiment of the disclosure;

FIG. 5(b) illustrates a block diagram indicating a communication of a video signal having a 3D endoscopic view of the actual surgical site from the local surgeon console to the remote surgeon console in accordance with an embodiment of the disclosure;

FIG. 5(c) illustrates a block diagram indicating a two-way transmission of audio and video data between conferencing systems at remote location and local operating room in accordance with an embodiment of the disclosure;

FIG. 6 illustrates steps for sending data from the remote surgeon console to the local surgeon console in accordance with an embodiment of the disclosure; and

FIG. 7 illustrates a flow diagram of the method for performing tele-surgery in accordance with an embodiment of the disclosure.

### DETAILED DESCRIPTION OF THE DISCLOSURE

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof. Throughout the patent specification, a convention employed is that in the appended drawings, like numerals denote like components.

Reference throughout this specification to “an embodiment”, “another embodiment”, “an implementation”, “another implementation” or similar language means that a particular feature, structure, or characteristic described in connection with the embodiment is included in at least one embodiment of the present disclosure. Thus, appearances of the phrase “in an embodiment”, “in another embodiment”, “in one implementation”, “in another implementation”, and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

Unless otherwise defined, all technical and scientific terms used herein have the same meaning as commonly understood by one of ordinary skill in the art to which this disclosure belongs. The device, system, and examples provided herein are illustrative only and not intended to be limiting.

The terms “a” and “an” herein do not denote a limitation of quantity, but rather denote the presence of at least one of the referenced items. Further, the term sterile barrier and sterile adapter denotes the same meaning and may be used interchangeably throughout the description.

Embodiments of the disclosure will be described below in detail with reference to the accompanying drawings.

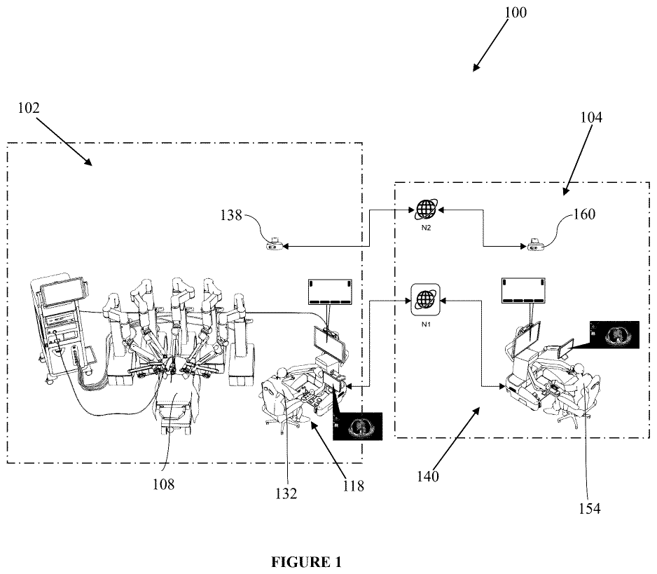

FIG. 1 illustrates an example implementation of multi-arm surgical robotic system (100) for tele-surgery in accordance with an embodiment of the disclosure. The system (100) comprises of a local system (102), a remote system (104), a private network (N1), and a public network (N2).

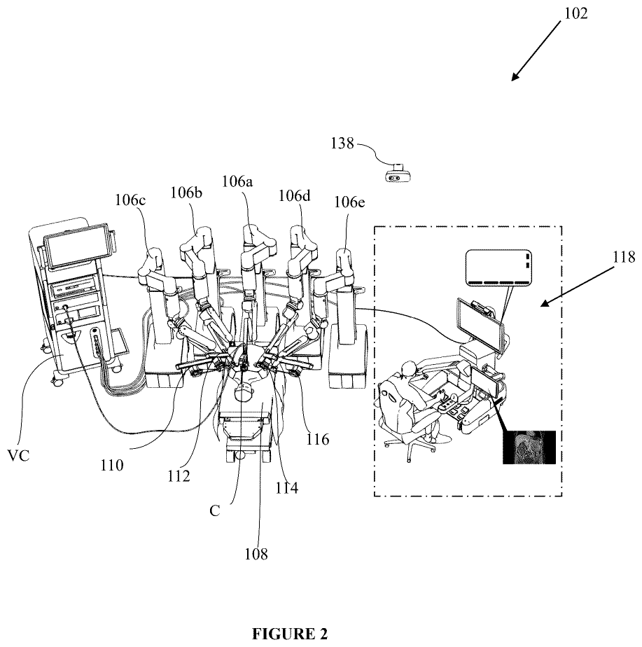

FIG. 2 illustrates an example implementation of a multi-arm teleoperated robotic surgical system kept at the location of surgery with one or more features in accordance with an embodiment of the disclosure. Specifically, FIG. 2 illustrates a local system comprising of a plurality of robotic arms (106a), (106b), (106c), (106d), (106d), (106d), (106e), each mounted on a robotic arm cart around an operating table (108). As an exemplary embodiment, a setup having five-robotic arms (106a), (106b), (106c), (106d), (106d), (106e), is depicted in FIG. 1. This depiction is for illustration purposes and the number of robotic arms may vary depending upon the type of surgery. The exemplary five robotic arms (106a), (106b), (106c), (106d), (106d), (106e), are arranged along the operating table (108) and may be arranged in different manner but not limited to the robotic arms (106a), (106b), (106c), (106d), (106d), (106e), arranged along the operating table (108). The robotic arms (106a), (106b), (106c), (106d), (106d), (106e), may be separately mounted on the five robotic arm carts or the robotic arms (106a), (106b), (106c), (106d), (106d), (106e), mechanically and/or operationally connected with each other or the robotic arms (106a), (106b), (106c), (106d), (106d), (106e), connected to a central body (not shown) such that the robotic arms (106a), (106b), (106c), (106d), (106d), (106e), branch out of a central body (not shown). One robotic arm out of the plurality of robotic arms (106a, 106b, 106c, 106d, 106e) is connected to an endoscopic camera (C), and the remaining robotic arms are each connected to one of a surgical robotic instruments (110, 112, 114, 116). Further, the local system comprises a local surgeon console (118), a vision cart (VC), a surgical instrument and accessory table (not shown), and a conferencing system (138).

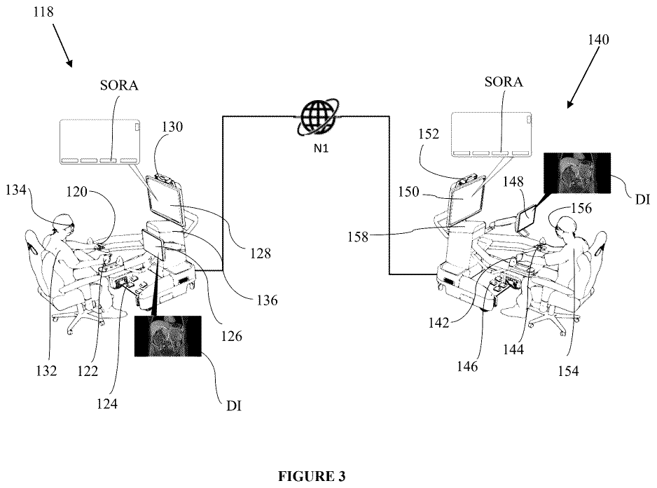

FIG. 3 illustrates an implementation of a local surgeon console connected to a remote surgeon console via a private network (N1) in accordance with an embodiment of the disclosure. The local surgeon console (118) comprises of a left-hand controller (120), a right-hand controller (122), foot pedals (124), a two-dimensional (2D) touch screen monitor (126), a three-dimensional (3D) HD monitor (128), and a head tracking camera (130) to track a movement of the head of a local surgeon (132) wearing a pair of trackable glasses (134), each coupled to a master controller (136), the master controller (136) provides control inputs to the plurality of robotic arms (106a, 106b, 106c, 106d, 106e). The local surgeon (132) takes control of the surgeon console (118). Further, a chair is provided for the local surgeon (132). The remote system (104) comprises of a remote surgeon console (140) and a conferencing system (160) (shown in FIG. 1). The remote surgeon console (140) comprises a left-hand controller (142), a right-hand controller (144), foot pedals (146), a two-dimensional (2D) touch screen monitor (148), a three-dimensional (3D) HD monitor (150), and a head tracking camera (152) to track a movement of the head of a remote surgeon (154) wearing a pair of trackable glasses (156), each coupled to a master controller (158), wherein the master controller (158) is operably coupled to the master controller (136) of the local surgeon console (118) via the private network (N1). Both the local surgeon console (118) and remote surgeon console (140) are identical. The remote surgeon (154) may be located at a distance from the actual operating room. The tele-surgery utilizes the private network (N1) to communicate data between a server and a client. The private network (N1) is a secure network with end-to-end encryption, and may be like a P2P, a MNLS, or a lease line. The local surgeon console (118) may act as a slave surgeon console and the remote surgeon console (140) acts as a master surgeon console. In this scenario the remote surgeon (154) takes the full control of the local surgeon console (118) (as shown in FIG. 1) to perform the tele-surgery.

The remote surgeon console (140) may utilize a portable chair to accommodate a remote surgeon (154) during a robotic tele-surgery. The essential components for controlling surgical robots are integrated in both the local surgeon console (118) and the remote surgeon console (140) to ensure a compact and user-friendly design. The remote surgeon console (140) may be utilized by an expert surgeon (154) sitting at a location which is far away from the local surgical operating room. In case of loss of telecommunication signal a local surgeon (132) sitting in the local operating room may take control. The local surgeon console (118) serves as the interface for the remote surgeon console (140) within the local operating room. Both surgeons (132) (154) respectively wear a pair of trackable 3D glasses (134) (156) to use with the respective surgeon console (118) (140). The surgeon's pair of trackable 3D glasses (134) (156) are tracked with a respective head tracking camera (130) (152) which can be secured to the respective 3D HD monitor (128) (150). Preferably, the head tracking camera (130) (152) can be secured to the top of the 3D HD monitor (128) (150). This is a safety feature to avoid distracted use of the multi-arm robotic surgical system (100) for tele-surgery and unintended motions while the surgeon's attention is not focused on the 3D HD Monitor (128) (150).

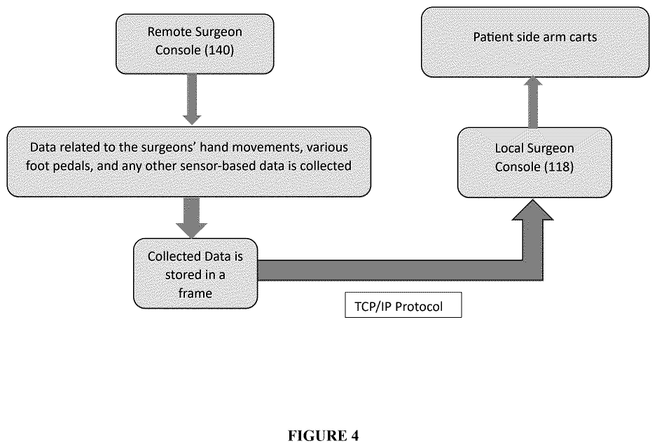

FIG. 4 illustrates a flow of communication of control input from the remote surgeon console (140) to the local surgeon console (118) located in the local operating room in accordance with an embodiment of the disclosure. The remote surgeon (154) sitting at a remote location uses hand controllers (142) (144) to perform a surgical action. All control input data related to movement of hand controllers (142) (144) by the remote surgeon (154), a data from various foot pedals (146), and any other sensor-based data for robotic surgical instruments (110, 112, 114, 116) is collected and communicated through a serial port to the master controller (158) of the remote surgeon console (140). A start byte and stop byte are added to the received data. The complete control input data thus received is stored in a frame in the master controller (158) of the remote surgeon console (140). The remote surgeon console (140) will act as a client and will send the data frame over a transmission network, which is a private network (N1) using TCP/IP communication protocol to the local surgeon console (118). The TCP/IP Communication protocol is a connection-oriented protocol which ensures the data is sent seamlessly between the client (remote surgeon console (140)) and the server (local surgeon console (118)). The local surgeon console (118) communicates with the patient side arms (106a, 106b, 106c, 106d, 106e). The received data enables the movement of different robotic surgical instruments (110, 112, 114, 116) utilized for surgery at the surgical site.

Both the conferencing system (138) of the local surgeon console (118) and the conferencing system (160) of the remote surgeon console (140), may have a processor (not shown), a camera (not shown), a 2D monitor (not shown), a sound device like a speaker (not shown) and a microphone (not shown). The local surgeon console (118) and the remote surgeon console (140) may feature an omnidirectional microphone and a radio station. These components work together to enable audio and video interaction between the local surgeon (132) at in the local operating room and the remote surgeon (154) at a remote location. The processor, the camera, the 2D monitor, the sound device, the microphone, and the radio station facilitate real-time communication using a public network (N2) as shown in FIG. 1, allowing the remote surgeon (154) to proctor and provide guidance and instructions to the local surgeon (132) during the surgery.

In place of simple trackable 3D glasses, a mixed reality headset holder consisting of an omnidirectional microphone (not shown) and a speaker (not shown) may be provided. The remote surgeon (154) may get an augmented environment with a pass-through or see-through feature for easy vision of the local operating room. The headset (not shown) may feature a virtual screen for viewing the endoscopic feed from the endoscope, a virtual control screen for manipulating chair ergonomics, a panel for patient details, a virtual screen for patient vitals monitoring, a virtual screen for remote proctoring access, and a virtual screen for robotic system controls. The headset may include a provision for 3D notifications for important troubleshooting and surgery status data.

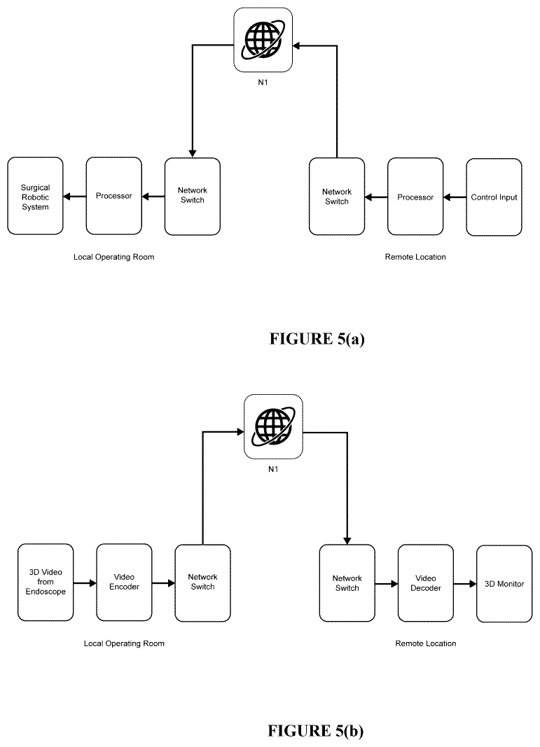

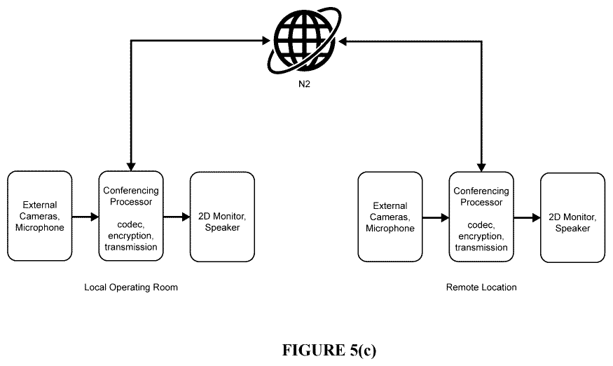

FIG. 5(a) illustrates a block diagram indicating a communication of a control input from a remote surgeon at a remote location to local operating room in accordance with an embodiment of the disclosure. The remote surgeon (154) provides control input which is transmitted to the master controller (136) of the local surgeon console (118) in the local operating room via the private network (N1). FIG. 5(b) illustrates a block diagram indicating a communication of a video signal having a 3D endoscopic view of the actual surgical site from the local surgeon console to the remote surgeon console in accordance with an embodiment of the disclosure. FIG. 5(c) illustrates a block diagram indicating a two-way transmission of audio and video data between conferencing systems at remote location and local operating room in accordance with an embodiment of the disclosure. The local surgeon (132) and the remote surgeon (154) may interact via the public network (N2).

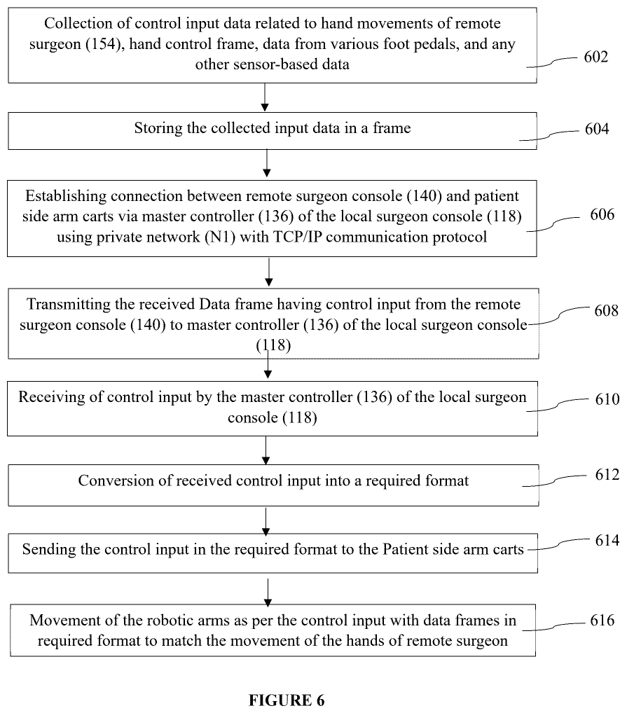

FIG. 6 illustrates steps for sending data from the remote surgeon console to the local surgeon console in accordance with an embodiment of the disclosure. Firstly, a control input data related to hand movements of the remote surgeon (154), hand control frame, data from various foot pedals, and any other sensor-based data is collected in step (602). The collected control input data is stored in a frame in step (604). Then, in step (606), a connection is established between the remote surgeon console (140) and the patient side arm carts via master controller (136) of the local surgeon console (118) using a private network (N1) with TCP/IP communication protocol. The received Data frame having the control input is transmitted from the remote surgeon console (140) to the master controller (136) of the local surgeon console (118) in step (608). The transmitted control input data is received by the master controller (136) of the local surgeon console (118) in step (610). The received control input data is converted into the required format in step (612). The control input data in the required format is sent to the Patient side arm carts in step (614). The movement of the robotic arms is done as per the control input with data frames in required format to match the movement of the hands of the remote surgeon (154) in step (616).

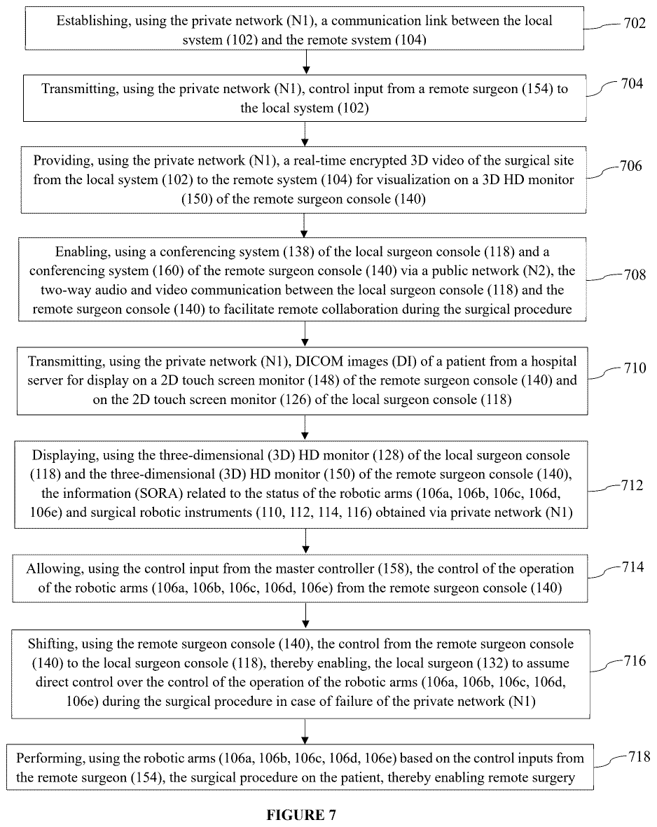

FIG. 7 illustrates a flow diagram of the method for performing tele-surgery in accordance with an embodiment of the disclosure. For the surgical procedure to be performed the robotic surgical instruments are finalized. The number of robotic arms is decided for the chosen robotic surgical procedure and preoperative planning is done. The intraoperative planning can be done as per discussion between the local surgeon (132) and the remote surgeon (154). In step (702), using the private network (N1), a communication link between the local system (102) and the remote system (104) is established. At step (704), using the private network (N1), a control input from a remote surgeon (154) is transmitted to the local system (102). In step (706), a real-time encrypted 3D video of the surgical site from the local system (102) is provided to the remote system (104) for visualization on a 3D HD monitor (150) of the remote surgeon console (140). Using the conferencing system (138) of the local surgeon console (118) and the conferencing system (160) of the remote surgeon console (140) via a public network (N2), a two-way audio and video communication is established between the local surgeon console (118) and the remote surgeon console (140) in step (708). This two-way audio and video communication facilitates remote collaboration during the surgical procedure. Using the private network (N1), DICOM images (DI) of a patient from a hospital server for display on a 2D touch screen monitor (148) of the remote surgeon console (140) and on the 2D touch screen monitor (126) of the local surgeon console (118), in the step (710). At step (712), the information (SORA) related to the status of the robotic arms (106a, 106b, 106c, 106d, 106e) and surgical robotic instruments (110, 112, 114, 116) is obtained from a hospital server via the private network (N1), and displayed using the three-dimensional (3D) HD monitor (128) of the local surgeon console (118) and the three-dimensional (3D) HD monitor (150) of the remote surgeon console (140). At step (714), the control of the operation of the robotic arms (106a, 106b, 106c, 106d, 106e) is allowed using the control input from the master controller (158) of the remote surgeon console (140). In step (716), the control from the remote surgeon console (140) may be shifted to the local surgeon console (118), which enables the local surgeon (132) to assume direct control over the control of the operation of the robotic arms (106a, 106b, 106c, 106d, 106e) during the surgical procedure in case of failure of the private network (N1). Remote surgery or tele-surgery is performed using the robotic arms (106a, 106b, 106c, 106d, 106e) based on the control inputs from the remote surgeon (154) in step (718).

In an example implementation of the present disclosure, the remote surgeon (154) at a remote location from the local operating room and the local surgeon (132) at the local operating room can swap the system control and perform the surgeries collaboratively. It is also possible that a part of the telesurgery procedure may be performed by the local surgeon (132) and the remaining procedure may be performed by the remote surgeon (154). Thus, the expertise of both the surgeons can be utilized in performing the tele-surgery.

In another exemplary embodiment, if the multi-arm surgical robotic system (100) for tele-surgery has a minimum of five robotic arms (106a, 106b, 106c, 106d, 106e), then the system (100) for tele-surgery can be utilized to serve as a dual console system, in which a partial control from the remote surgeon console (140) can be shifted to the local surgeon console (118). Then, the expert surgeon/remote surgeon (154) may operate any three arms out of the five robotic arms (106a, 106b, 106c, 106d, 106e) and the remaining arms can be operated by the local surgeon (132) acting as an assistant surgeon.

The present disclosure has the following advantages: Both the local surgeon console (118) and the remote surgeon console (140) of the present disclosure are portable and have an ergonomic design. Further, the surgeon's comfort and control during robotic surgery is enhanced. The integration of essential components is compact. A precise instrument control is obtained through the use of various sensors. The multi-arm robotic surgical system (100) for tele-surgery can also be utilized for tele-proctoring. Further, in any adverse scenario where there is loss of communication (due to any failure in transmission network (N)) between the remote surgeon console (140) and the local surgeon console (118), the local surgeon console (118) can take over whole control of the surgical procedure. Moreover, the system (100) can be utilized in a way, such that the remote surgeon (154) may operate any three arms out of the five arms and the remaining arms may be operated by the local surgeon (132), who may act as an assistant surgeon being present in the local operating room.

The foregoing descriptions of exemplary embodiments of the present disclosure have been presented for purposes of illustration and description. They are not intended to be exhaustive or to limit the disclosure to the precise forms disclosed, and obviously many modifications and variations are possible in light of the above teaching. The exemplary embodiment was chosen and described in order to best explain the principles of the disclosure and its practical application, to thereby enable others skilled in the art to best utilize the disclosure and various embodiments with various modifications as are suited to the particular use contemplated. It is understood that various omissions, substitutions of equivalents are contemplated as circumstance may suggest or render expedient but is intended to cover the application or implementation without departing from the spirit or scope of the claims of the present disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the disclosure, any limitations arising on account of the same are not intended. As would be apparent to a person in the art, various working modifications may be made to the apparatus in order to implement the inventive concept as taught herein.

We claim:

1. A multi-arm surgical robotic system for tele-surgery, comprising a local system, a remote system, a private network, and a public network characterized in that:
the local system comprises:
a plurality of robotic arms arranged along an operating table, wherein a robotic arm out of the plurality of robotic arms is connected to an endoscopic camera, and the remaining robotic arms are each connected to one of a surgical robotic instruments;
a local surgeon console comprising a left-hand controller, a right-hand controller, foot pedals, a two-dimensional touch screen monitor, a three-dimensional HD monitor, and a head tracking camera to track a movement of the head of a local surgeon wearing a pair of trackable glasses, each coupled to a master controller, the master controller provides a control input received from the local surgeon to the plurality of robotic arms; and
a conferencing system,
the remote system comprises:
a remote surgeon console comprising a left-hand controller, a right-hand controller, foot pedals, a two-dimensional touch screen monitor, a three-dimensional HD monitor, and a head tracking camera to track a movement of the head of a remote surgeon wearing a pair of trackable glasses, each coupled to a master controller, wherein the master controller is operably coupled to the master controller of the local surgeon console via the private network; and
a conferencing system operably connected to the conferencing system via the public network;
the private network is configured to:
transmit the control input provided by the remote surgeon to the master controller of the local surgeon console, and transmit a real-time, encrypted 3D video stream of the surgical site from the local surgeon console to the three-dimensional HD monitor of the remote surgeon console;
retrieve DICOM images related to a patient from a hospital server connected to the private network and provide them to the master controller for displaying on the 2D display of the remote surgeon console and the master controller for displaying on the 2D display of the local surgeon console; and
transmit the information related to the surgical instruments and the status of each robotic arms to the master controller to be displayed on the three-dimensional HD monitor,
the public network is configured to facilitate a two-way transmission of real-time audio and video data between the conferencing system of the remote surgeon console and the conferencing system of the local surgeon console;
wherein the communication between the local surgeon console and the remote surgeon console via the private network, enables the remote surgeon to control and monitor the robotic arms, allowing the remote surgeon to perform a robotic surgical procedure remotely,
wherein the control from the remote surgeon console can be shifted to the local surgeon console, allowing the local surgeon to assume control of the robotic surgical procedure in case of failure of the private network.

2. The system as claimed in claim 1, wherein in the multi-arm surgical robotic system having a minimum of five robotic arms, a partial control from the remote surgeon console can be shifted to the local surgeon console, enabling the remote surgeon to operate any three arms out of the five or more robotic arms and the remaining arms can be operated by the local surgeon acting as an assistant surgeon.

3. The system as claimed in claim 1, wherein the left-hand controller, the right-hand controller, and the foot pedals are configured to capture the control input from the local surgeon.

4. The system as claimed in claim 1, wherein the two-dimensional touch screen monitor coupled to the master controller is configured to serve as a graphical user interface to capture the control input from the local surgeon.

5. The system as claimed in claim 1, wherein the real-time 3D video stream of the surgical site is displayed on the three-dimensional HD monitor of the local surgeon console.

6. The system as claimed in claim 1, wherein the left-hand controller, the right-hand controller, and the foot pedals are configured to receive the control input from the remote surgeon.

7. The system as claimed in claim 1, wherein the two-dimensional touch screen monitor coupled to the master controller is configured to be used as a graphical user interface to capture the control input from the remote surgeon.

8. The system as claimed in claim 1, wherein each conferencing system comprises of a processor, a camera, a 2D monitor, a speaker, and a microphone.

9. The system as claimed in claim 1, wherein the real-time two-way video data transmitted between the conferencing system of the remote surgeon console and the conferencing system of the local surgeon console can be displayed on separate 2D monitors at the remote location and the local operating room.

10. A method of tele-surgery using a multi-arm surgical robotic system comprising a local system, a remote system, a private network, and a public network, the method comprising:
establishing, using the private network, a communication link between the local system and the remote system, wherein the local system includes a plurality of robotic arms and a local surgeon console, and the remote system includes a remote surgeon console;
transmitting, using the private network, a control input from a remote surgeon to the local system, wherein the control input controls the movement and operation of the plurality of robotic arms;
providing, using the private network, a real-time encrypted 3D video of the surgical site from the local system to the remote system for visualization on a 3D HD monitor of the remote surgeon console;
enabling, using a conferencing system of the local surgeon console and a conferencing system of the remote surgeon console via a public network, the real-time two-way audio and video communication between the local surgeon console and the remote surgeon console to facilitate remote collaboration during the surgical procedure;
transmitting, using the private network, DICOM images of a patient from a hospital server for display on a 2D touch screen monitor of the remote surgeon console and on the 2D touch screen monitor of the local surgeon console;
displaying, using the three-dimensional HD monitor of the local surgeon console and the three-dimensional HD monitor of the remote surgeon console, the information related to the status of the robotic arms and surgical robotic instruments);
allowing, using the control input from the master controller, the control of the operation of the robotic arms from the remote surgeon console, wherein the remote surgeon is located distantly from the patient;
shifting, using the remote surgeon console, the control from the remote surgeon console to the local surgeon console, thereby enabling, the local surgeon to assume direct control over the control of the operation of the robotic arms during the surgical procedure in case of failure of the transmission network; and
performing, using the robotic arms based on the control input from the remote surgeon, the surgical procedure on the patient, thereby enabling remote surgery.

11. The method as claimed in claim 10, wherein in the multi-arm surgical robotic system having a minimum of five robotic arms, a partial control from the remote surgeon console can be shifted to the local surgeon console, enabling the remote surgeon to operate any three arms out of the plurality of robotic arms and the remaining arms can be operated by the local surgeon acting as an assistant surgeon.
